(19) United States
(12) Patent Application Publication
(10) Pub. No.: US 2010/0003762 A1
(43) Pub. Date: Jan. 7, 2010

(54) Permanent chemical marker and identification of information in polymers

(76) Inventors: Axel Kolbe, Neundorf, (DE); Hardy Markwitz, Rudolstadt (DE)

Correspondence Address: PROPAT, L.L.C., 425-C SOUTH SHARON AMITY ROAD, CHARLOTTE, NC, 28211-2841, US

(21) Appl. No.: 12/446,770

(22) PCT Filed: Oct. 23, 2007
(86) PCT No.: PCT/DE2007/001906

§ 371 Date: Apr. 23, 2009

(30) Foreign Application Priority Data
Oct. 27, 2006 (DE) 10 2006 051 388.6

(51) Int. Cl. G01N 33/44; C08F 283/02

(52) U.S. Cl. 436/85; 525/437

(57) ABSTRACT

The invention relates to a process for chemical marking and identification of information in polymers and additives, such as ageing additives, thermal stabilizer additives, light stabilizer additives, plasticizer additives or flame retardant additives and dyes and other high-value additives. The marking is effected by the use of defined atomic masses in the form of isotopes of suitable elements in chemical compounds which are incorporated into the polymer matrix and anchored during the production. The result is a chemical code which can be recalled over the entire product life without adversely affecting the properties of the polymer through high concentrations of extraneous substances. It is possible by this process to trace back the origin of a polymer, to detect possible mixtures with chemically identical products from other manufacturers, or to clarify at a later stage whether an additive has been added in the concentration specified.

The invention relates to a process for the permanent chemical marking and identification of information in polymers, and also to polymers thus marked.

Rare earth metals (elements 57 to 71), and also hafnium and tantalum, are not industrially relevant for the production of polymers and of polymer additives. Only cerium and lanthanum are bound in the solid phase when they are used as catalysts. It is known that the stable or very long-lived isotopes of the compounds mentioned cover the entire range from 139 to 181 amu (atomic mass unit) (Ullmann's Encyclopedia of Industrial Chemistry (5th edition), VCH 1993, volume A 22, pp. 607ff). It can be assumed that none of these elements is not detected in industrial polymers or in polymer additives. Nor do they have widespread distribution, and analyses of said elements are unlikely to give blind values deriving from the operating environment of the test laboratory. It is moreover known that modern analysis methods, particularly ICPMS, can be used to determine concentrations extending as far as 0.1 μg/kg of the rare earth metals.

There have been repeated attempts to achieve marking of polymers. As early as 1969, U.S. Pat. No. 3,439,168 described a process based on the measurement of additives comprising gold, indium, or lanthanum, using neutron activation analysis and gamma-ray analysis.

JP2002 332414 discloses a method describing all of the elements from gallium to radium as possible marking elements. The operating range of the process, from 0.1 to 1000 ppm, is very high, because the only measurement methods utilized are based on interaction with the electron shell of the analyte. The first problem is economic: the costs for markings can be of the order of magnitude of the costs of the material itself, or can even exceed these, because some of the elements are extremely rare. By way of example, marking of 100 000 tons of polymer with, for example, 10 mg/kg of ruthenium requires 1000 kg of ruthenium, and this corresponds to 8 times the global annual production amount of 120 kg/year. Furthermore, it is impossible to be certain that these high admixtures of up to 0.1% of foreign substances do not have adverse effects on the properties of the product.

WO2005/054132 describes the use of inert rare earth metal compounds for marking purposes. These are incorporated in suspended form into the polymer matrix. The materials involved are oxides, sulfides, borides, halides, silicides, and specific mention is also made of acetate, carbonate, hydroxide, nitrate, oxalate, sulfate, and alkyl compounds. The thermodynamic stability of alkyl compounds of rare earth metals is not entirely satisfactory, because of the chemical properties of said elements. In summary, the aim is always to achieve inclusions of rare-earth-metal particles which are inert toward the polymer matrix. This approach carries the risk of inhomogeneity of distribution within the matrix, particularly at low concentrations. The operating range, from 1 to 20 000 mg/kg, is very high, and compounds in this concentration range can have an adverse effect on the chemical and physical properties of polymers. There is also an economic problem: the costs for marking can be of the order of magnitude of the costs of the material itself, or can even exceed these, because the elements are extremely rare. By way of example, addition of 50 mg/kg of europium (about ε17 000/kg) would increase the cost of 1 ton of polymer by about ε850. The availability of the raw materials is in some cases very restricted and has not been sufficiently considered, since at a concentration of 50 mg/kg 1 kg of europium would be sufficient only for 20 tons of polymer. On the basis of the chemical variety of the polymers to be protected, it can be concluded that insufficient attention has been paid to the migration behavior of the rare earth metal compounds. Furthermore, the use of detection principles based solely on electromagnetic waves, i.e. on interactions between the electron shell of the rare earth metals and radiation, provides insufficient sensitivity. An alternative is the use of an inductively coupled plasma with a mass-selective detector (ICPMS).

According to WO 01/27699, banknotes can be protected from counterfeiting by addition of lanthanide chelates. The detection methods relate to the UV/VIS region. However, this method cannot be used to store any information in a polymer.

Numerous publications, e.g. EP 1 356 478 (=U.S. Pat. No. 6,790,542 B2), EP 1 409 997 (=U.S. Pat. No. 6,514,617 B), EP 1 154 990, or US 2005/095715, concern the marking of materials. However, none of the specifications gives any detail as to how multilayered information can be encoded, going beyond the simple abundance of elements and compounds. All of the approaches assume that an anti-counterfeiting marking can be achieved simply by adding rare elements. Insufficient attention is paid here to the fact that within the target concentration range analysis is possible by using even simple analysis methods, such as calorimetric or photometric methods. The protection can therefore easily be circumvented. Furthermore, protection of wide variety of chemical products is not achievable, since the range of information is too small. The necessary range of data and protection from counterfeiting is achievable only by the linking of chemical additives with a mathematical coding system. No publication has hitherto given any details of this linking.

CH 586 255 describes a marking process in which an amount of from 0.001 to 10 ppm of a mixture composed of lead and lanthanum is added to a polyester. A marked polyester is identified by detection of lanthanum and lead through neutron activation analysis and, respectively, atomic absorption spectroscopy.

CH 586 733 A5 discloses a marking process for polyamides and moldings produced therefrom. Here, mixtures of lanthanum compounds, lead compounds, and zinc compounds are added to the polyamide during production, where lead content is from 1 to 30 ppm, zinc content is from 2 to 50 ppm, and lanthanum content is from 0.1 to 30 ppm. The proportions of lead, zinc and lanthanum present in the polyamide are determined by means of atomic absorption spectroscopy or neutron activation analysis.

It was an object of the invention to develop an economically efficient process which can store information in polymers and in their additives, for example aging additives, heat-stabilizer additives, light-stabilizer additives, plasticizer additives or flame retardant additives, or else dyes and other high-specification additives, in order to render the same unambiguously distinguishable from chemically identical polymers and polymer additives from other producers. The intention was to find a chemical coding system which can be read over the entire product life of a polymer, without any adverse effect on the properties of the polymer caused by high concentrations of foreign substances. The intention is that said process can trace the source of a polymer, prove possible mixing with chemically identical products from other producers, or provide subsequent clarification as to whether an additive has been added at the specified concentration.

The object is achieved via a process for the permanent chemical marking and identification of polymers, encompassing the steps of
coding, where the polymer receives additions of at least three different isotopes, selected from the group of the natural or synthetic stable isotopes with atomic weight of 89, 93, 103, 107, 115, 127, 133, 139-181, 193, 197, 205, 209, and 238 amu (atomic mass unit), in a defined ratio of amounts, in each case amounting to not more than 50 μg/kg, preferably not more than 20 μg/kg, and particularly preferably in the range from 0.1 to 3 μg/kg or from 6 to 20 μg/kg, based on the total weight of the polymer, and
identification, where the isotopes present in the polymer are determined by using solution chemistry to denature the material, and using inductively coupled plasma with mass spectroscopy (ICPMS).

This means that the information from the producer is converted into a placealized code, and that this code is coupled with a code of various chemical isotopes, where various concentration ranges can be allocated to the isotopes. For this, one place in the mathematical code is allocated to selected isotopes of chemical elements. The code for the marking process has at least three places, and an atomic weight of 89, 93, 103, 107, 115, 127, 133, 139 to 181, 193, 197, 205, 209 or 238 amu is unambiguously allocated to each of these places, and the amount added to the polymer of the isotope with this atomic weight is calculated from the value of the character occupying that place.

The abovementioned isotopes are particularly effective for avoiding problems. These, in rising order, are provided with numerals which give their position within the code, i.e. 1-89 amu, 2-93 amu, 3-103 amu, 4-107 amu, 5-115 amu, 6-127 amu, 7-133 amu, 8-139 amu, . . . , 50-181 amu, 51-193 amu, 52-197 amu, 53-205 amu, 54-209 amu and 55-238 amu. The 55 places allow the global code to be used for deriving a wide variety of subsystems (licenses), utilizing a defined sequence of places in the code.

The information can also be coded via concentration levels. A mathematical trinary system can be coded using allocation to the concentration ranges zero-low-high. In a trinary number system, for example, level A can correspond to a concentration of <0.1 μg/kg, level B to a concentration of 1 μg/kg and level C to 10 μg/kg. Solution chemistry denaturing of the polymer, followed by measurement by means of ICPMS permits detection of the concentration range >0.1 μg/kg. An advantage of concentration levels is that the rising measurement error in the vicinity of the detection limit is taken into account. The result is a marked decrease in the probability of incorrect interpretation. The following factors make it more difficult for third parties to evaluate the test analysis, and for other producers to imitate the product: the 55-place code, the number of elements underlying the isotopes and the availability of these elements, and the isotope ratio, which is either natural or artificially altered by isotope enrichment, e.g. through ion fractionation.

The isotope levels can moreover by utilized in order to encode not only a producer signature but also production data, for example production plant, quarter in which production took place, brief batch identifier. Within the global code, the system provides various security levels. By way of example, a signature using a total of 12 μg/kg of readily available isotopes is economically acceptable even for standard polymers, since 12 g of the isotopes are theoretically sufficient for 1000 tons. For very sensitive applications, use of isotope-enriched elements based on neodymium, samarium, gadolinium, dysprosium, erbium, ytterbium, or hafnium is a rational choice. The shift in the natural concentration ratio of the isotopes acts like an unmistakable fingerprint, since the artificially generated isotope levels are very difficult to copy. Very rare isotopes, such as 151 amu or 153 amu europium or 103 amu rhenium, can likewise be rational constituents where codes have to meet very high demands. If the total concentration of isotopes used is 25 μg/kg, with a cost factor of ε40 000/kg of isotopes, the increased cost for chemical coding need be only ε1/ton of polymer. Dilution does not cause loss of the information, which can be detected on the basis of the ratios of the isotopes to one another, and the concentration levels.

The isotopes are incorporated in the form of chemical compounds of said isotopes into the polymer matrix. These chemical compounds can be salts of the isotope-forming elements, or monomers modified with the element, these being monomers of the polymers to be marked, or element-containing organic compounds having functional groups, where these preferably have a very little tendency toward migration within the polymer to be marked. The most rational strategy appears to be to form adducts of the isotopes (except for 127 amu) onto acid structures, such as carboxy groups, sulfonic acid groups, or phosphonic acid groups. Binding of the isotopes into suitable chemical compounds which are similar to the monomer permits the code to be incorporated into the main chain, or to be anchored securely within the polymer via a suitable anchoring group. Another rational method alongside this method is the use of chelating agents and of surfactant-type structures, which provide a migration-inhibiting effect by way of hydrophobic chains.

The code for the marking process therefore has 55 places (t₁; t₂; . . . ; t₅₅), where an atomic weight is allocated to each place of the trinary code in rising order: t₁-89 amu, t₂-93 amu, t₃-103 amu, t₄-107 amu, t₅-115 amu, t₆-127 amu, t₇-133 amu, t₈-139 amu . . . t₅₀-181 amu, t₅₁-193 amu, t₅₂-197 amu, t₅₃-205 amu, t₅₄-209 amu and t₅₈-238 amu, and the amount added to the polymer of the isotope with this atomic weight is either a) zero or an amount below the detection limit, b) from 0.1 to 3 μg/kg, or c) from 6 to 20 μg/kg. The isotopes are added prior to or during the production of the polymer, if appropriate blended with a polymer additive. It is also possible that a masterbatch is admixed with the polymer and comprises salts of the isotopes, the amount present of these preferably being greater than 2% by weight, based on the total weight of the masterbatch.

The information is preferably identified by using solution-chemistry methods for denaturing and mineralization of the polymer or of the polymer additive, and using ICPMS to determine the concentration of the isotopes. The producer can use the test report to read the code and compare it with the original data, but completion of this task does not require that any knowledge of the structure of the signature and of the code be passed to the test system. Quantification to confirm whether a sufficient amount of an additive has been added is also possible as long as at least one isotope is added at a concentration which generates level C (=10 μg/kg) in the final product. This concentration is quantifiable with a sufficiently small error to permit checking of the amount added.

If a masterbatch is used, comprising the chemical code in concentrated form, the resource required for the marking process during production is merely that for adding the required concentration of the masterbatch. Concentrations used here are preferably more than 2% of coding salts. Contract manufacture can thus also be included in the coding system, without any need to provide the subcontractor with information about the security system.

If the basis utilized from a data comprises the isotopes with weight of 89, 93, 103, 107, 115, 127, 133, 139-181, 193, 197, 205, 209, and 238 AMU, the code can be trinary, having 55 places, with 1.7*10²⁶ solutions. The result is chemical marking which can be read over the entire product life of a polymer without any adverse effect on the properties of the polymer caused by high concentrations of foreign substances. The intention is that said process can trace the source of a polymer, prove possible mixing with chemically identical products from other producers, or provide subsequent clarification as to whether an additive has been added at the specified concentration.

The polymers marked by the process of the invention comprise at least three isotopes, selected from the group of the natural or synthetic stable isotopes with atomic weight of 89, 93, 103, 107, 115, 127, 133, 139 to 181, 193, 197, 205, 209, and 238 AMU, where the proportion by weight of an isotope is in each case not more than 50 μg/kg, preferably not more than 20 μg/kg, based on the total weight of the polymer.

It is preferable that the polymer of the invention comprises in each case, based on the total weight of the polymer, an amount of from 0.1 to 3 μg/kg or from 6 to 20 μg/kg of the isotopes used for the marking process.

In further embodiments of the invention, the isotopes are present in the form of the following chemical compounds within the polymer:
salts of the isotopes;
organic compounds of the isotopes which have functional groups, in particular carboxy groups, sulfonic groups, or phosphonic groups, preference being given here to organic compounds whose molecules have very little tendency toward migration in the polymer to be marked;
organic compounds of the isotopes which comprise modified monomeric units of the polymers to be marked; and
compounds of the isotopes which are chelating agents or which have surfactant-like structures.

### EXAMPLE 1

The following were admixed with a polyethylene terephthalate:

10 μg/kg of isotope 181 amu (10 μg of tantalum in the form of an organic tantalum salt),
1 μg/kg of isotope 146 amu (6 μg of neodymium in the form of an organic neodymium salt),
1 μg/kg of isotope 89 (1 μg of yttrium in the form of an organic yttrium salt), and
10 μg/kg of isotope 93 (10 μg of niobium in the form of an organic niobium salt).

The material was denatured using a microwave-assisted solution-chemistry method and analyzed using ICPMS. The measured values obtained are found in Table 1.

The license includes a producer code (Table 2) composed of places 15 (isotope 146), 38 (isotope 169), and 50 (isotope 181) of the code, with the information BAC, and the information can be determined from the measured results. The third quarter of the year A has also been codified as production time with code BC, according to the key to quarters of the year A/B.

TABLE 1
Analysis results and coding for Example 1

| Code place | 1 | 2 | 3 | 4 | 5 | 6 | 7 | 8 | 9 | 10 | 11 |
|---|---|---|---|---|---|---|---|---|---|---|---|
| Isotope (amu) | 89 | 93 | 103 | 107 | 115 | 127 | 133 | 139 | 140 | 141 | 142 |
| Measured value (μg/kg) | 0.8 | 9.7 | <0.1 | <0.1 | <0.1 | <0.1 | <0.1 | <0.1 | <0.1 | <0.1 | 1.6 |
| Code | B | C | A | A | A | A | A | A | A | A | B |
| Code place | 12 | 13 | 14 | 15 | 16 | 17 | 18 | 19 | 20 | 21 | 22 |
| Isotope (amu) | 143 | 144 | 145 | 146 | 147 | 148 | 149 | 150 | 151 | 152 | 153 |
| Measured value (μg/kg) | 0.8 | 1.7 | 0.3 | 1.1 | <0.1 | 0.2 | <0.1 | <0.1 | <0.1 | <0.1 | <0.1 |
| Code | B | B | B | B | A | B | A | A | A | A | A |
| Code place | 23 | 24 | 25 | 26 | 27 | 28 | 29 | 30 | 31 | 32 | 33 |
| Isotope (amu) | 154 | 155 | 156 | 157 | 158 | 159 | 160 | 161 | 162 | 163 | 164 |
| Measured value (μg/kg) | <0.1 | <0.1 | <0.1 | <0.1 | <0.1 | <0.1 | <0.1 | <0.1 | <0.1 | <0.1 | <0.1 |
| Code | A | A | A | A | A | A | A | A | A | A | A |
| Code place | 34 | 35 | 36 | 37 | 38 | 39 | 40 | 41 | 42 | 43 | 44 |
| Isotope (amu) | 165 | 166 | 167 | 168 | 169 | 170 | 171 | 172 | 173 | 174 | 175 |
| Measured value (μg/kg) | <0.1 | <0.1 | <0.1 | <0.1 | <0.1 | <0.1 | <0.1 | <0.1 | <0.1 | <0.1 | <0.1 |
| Code | A | A | A | A | A | A | A | A | A | A | A |
| Code place | 45 | 46 | 47 | 48 | 49 | 50 | 51 | 52 | 53 | 54 | 55 |
| Isotope (amu) | 176 | 177 | 178 | 179 | 180 | 181 | 193 | 197 | 205 | 209 | 238 |
| Measured value (μg/kg) | <0.1 | <0.1 | <0.1 | <0.1 | <0.1 | 7.9 | <0.1 | <0.1 | <0.1 | <0.1 | <0.1 |
| Code | A | A | A | A | A | C | A | A | A | A | A |

TABLE 2
Sub-coding for Example 1

| Quarter | I/A | II/A | III/A | IV/A | I/B | II/B | III/B | IV/B |
|---|---|---|---|---|---|---|---|---|
| Isotope 89 | 1 | 1 | 1 | 10 | 10 | 10 | <0.1 | <0.1 |
| (μg/kg) | | | | | | | | |
| Code | B | B | B | C | C | C | A | A |
| Isotope 93 | <0.1 | 1 | 10 | <0.1 | 1 | 10 | 1 | 10 |
| (μg/kg) | | | | | | | | |
| Code | A | B | C | A | B | C | B | C |

### EXAMPLE 2

The intention is exclusive provision of a security feature to a PEEK engineering plastic. 1.1 μg/kg of isotope 174 amu (2.1 μg of isotope-enriched ytterbium in the form of an organic ytterbium salt) was admixed with the material. The available 112 mg of synthetic ytterbium isotope mixture were sufficient for about 50 tons of polymer. The isotope distribution of 168, 170, 171, 172, 173, 174 and 176 allowed it to be distinguished unambiguously from natural ytterbium, the constitution of which is 168-0.13%, 170-3.04%, 171-14.28%, 172-21.83%, 173-16.13%, 174-31.83%, and 176-12.76%. The measured values obtained were as follows (see Table 3):

TABLE 3
Analysis results and coding for Example 2

| Code place | 1 | 2 | 3 | 4 | 5 | 6 | 7 | 8 | 9 | 10 | 11 |
|---|---|---|---|---|---|---|---|---|---|---|---|
| Isotope (amu) | 89 | 93 | 103 | 107 | 115 | 127 | 133 | 139 | 140 | 141 | 142 |
| Measured value (μg/kg) | <0.1 | <0.1 | <0.1 | <0.1 | <0.1 | <0.1 | <0.1 | <0.1 | <0.1 | <0.1 | <0.1 |
| Code | A | A | A | A | A | A | A | A | A | A | A |
| Code place | 12 | 13 | 14 | 15 | 16 | 17 | 18 | 19 | 20 | 21 | 22 |
| Isotope (amu) | 143 | 144 | 145 | 146 | 147 | 148 | 149 | 150 | 151 | 152 | 153 |
| Measured value (μg/kg) | <0.1 | <0.1 | <0.1 | <0.1 | <0.1 | <0.1 | <0.1 | <0.1 | <0.1 | <0.1 | <0.1 |
| Code | A | A | A | A | A | A | A | A | A | A | A |
| Code place | 23 | 24 | 25 | 26 | 27 | 28 | 29 | 30 | 31 | 32 | 33 |
| Isotope (amu) | 154 | 155 | 156 | 157 | 158 | 159 | 160 | 161 | 162 | 163 | 164 |
| Measured value (μg/kg) | <0.1 | <0.1 | <0.1 | <0.1 | <0.1 | <0.1 | <0.1 | <0.1 | <0.1 | <0.1 | <0.1 |
| Code | A | A | A | A | A | A | A | A | A | A | A |
| Code place | 34 | 35 | 36 | 37 | 38 | 39 | 40 | 41 | 42 | 43 | 44 |
| Isotope (amu) | 165 | 166 | 167 | 168 | 169 | 170 | 171 | 172 | 173 | 174 | 175 |
| Measured value (μg/kg) | <0.1 | <0.1 | <0.1 | <0.1 | <0.1 | <0.1 | <0.1 | 0.1 | 0.4 | 0.9 | <0.1 |
| Code | A | A | A | A | A | A | A | A | B | B | A |
| Code place | 45 | 46 | 47 | 48 | 49 | 50 | 51 | 52 | 53 | 54 | 55 |
| Isotope (amu) | 176 | 177 | 178 | 179 | 180 | 181 | 193 | 197 | 205 | 209 | 238 |
| Measured value (μg/kg) | 0.7 | <0.1 | <0.1 | <0.1 | <0.1 | <0.1 | <0.1 | <0.1 | <0.1 | <0.1 | <0.1 |
| Code | B | A | A | A | A | A | A | A | A | A | A |

1. A process for the permanent chemical marking and identification of polymers, comprising the steps of
coding, said coding comprising adding at least three different isotopes to the polymer, said isotopes selected from the group of the natural or synthetic stable isotopes with atomic weight of 89, 93, 103, 107, 115, 127, 133, 139-181, 193, 197, 205, 209, and 238 AMU (atomic mass unit), in a defined ratio of amounts, in each case amounting to not more than 50 μg/kg, based on the total weight of the polymer, and
identifying, said identifying comprising determining the isotopes present in the polymer by denaturing the polymer using solution chemistry, and performing inductively coupled plasma with mass spectroscopy (ICPMS).

2. The process as claimed in claim 1, wherein said identifying further comprises determining the concentration of isotopes.

3. The process as claimed in claim 1, wherein the amounts added of the isotopes in each case amount to not more than 20 μg/kg, based on the total weight of the polymer.

4. The process as claimed in claim 1, wherein the proportion by weight added of the isotopes is in each case in the range from 0.1 to 3 μg/kg or from 6 to 20 μg/kg, based on the total weight of the polymer.

5. The process as claimed in claim 1, wherein the code for the marking process has at least three places, and an atomic weight of 89, 93, 103, 107, 115, 127, 133, 139 to 181, 193, 197, 205, 209 or 238 amu is unambiguously allocated to each of these places, and the amount added to the polymer of the isotope with this atomic weight corresponds to a predetermined concentration level.

6. The process as claimed in claim 1, wherein the code for the marking process is trinary and has 55 places (t₁; t₂; . . . ; t₅₅), where an atomic weight is allocated to each place of the trinary code in rising order: t₁-89 amu, t₂-93 amu, t₃-103 amu, t₄-107 amu, t₅-115 amu, t₆-127 amu, t₇-133 amu, t₈-139 amu . . . t₅₀-181 amu, t₅₁-193 amu, t₅₂-197 amu, t₅₃-205 amu, t₅₄-209 amu and t₅₈-238 amu, and the amount added to the polymer of the isotope with this atomic weight is either a) zero or an amount below the detection limit, b) from 0.1 to 3 μg/kg, or c) from 6 to 20 μg/kg.

7. The process as claimed in claim 1, wherein the isotopes are added prior to or during the production of the polymer, optionally blended with a polymer additive.

8. The process as claimed in claim 1, wherein a masterbatch is admixed with the polymer and said masterbatch comprises salts of the isotopes.

9. A polymer marked according to claim 1, said polymer comprising at least three isotopes, selected from the group of the natural or synthetic stable isotopes with atomic weight of 89, 93, 103, 107, 115, 127, 133, 139 -181, 193, 197, 205, 209, and 238 AMU (atomic mass unit), where the proportion by weight of an isotope is in each case not more than 50 μg/kg, based on the total weight of the polymer.

10. The polymer as claimed in claim 9, wherein the amount of an isotope is in each case not more than 20 μg/kg, based on the total weight of the polymer.

11. The polymer as claimed in claim 9, wherein the amount of the isotopes is in each case from 0.1 to 3 μg/kg or from 6 to 20 μg/kg, based on the total weight of the polymer.

12. The polymer as claimed in claim 9, wherein the polymer comprises salts of the isotopes.

13. The polymer as claimed in claim 9, wherein the polymer comprises organic compounds of the isotopes having functional groups.

14. The polymer as claimed in claim 9, wherein the polymer comprises organic compounds comprised of modified monomeric units of the polymers to be marked with the isotopes.

15. The polymer as claimed in claim 9, wherein the polymer comprises compounds of the isotopes which involve chelating agents or surfactant.

16. The process as claimed in claim 8, wherein the masterbatch comprises salts of the isotopes in an amount of greater than 2% by weight, based on the total weight of the masterbatch.

17. The polymer as claimed in claim 13, wherein said polymer comprises organic compounds of the isotopes having carboxy groups, sulfonic groups or phosphonic groups.

18. The polymer as claimed in claim 13, wherein said polymer comprises organic compounds of the isotope having little tendency toward migration in the polymers.
